(19) United States
(12) Patent Application Publication
(10) Pub. No.: US 2026/0206810 A1
(43) Pub. Date: Jul. 23, 2026

(54) NATURAL SWEETENER COMPOSITIONS AND USES THEREOF

(71) Applicant: Firmenich SA, Satigny (CH)

(72) Inventors: Wolfgang FIEBER, Ornex (FR); Pascal BEAUSSOUBRE, COLLONGES SOUS SALEVE (FR)

(73) Assignee: Firmenich SA, Satigny (CH)

(21) Appl. No.: 19/140,482

(22) PCT Filed: Dec. 7, 2023
(86) PCT No.: PCT/EP2023/084678

§ 371 Date: Jun. 18, 2025

(30) Foreign Application Priority Data
Dec. 22, 2022 (EP) 22215736.4

(51) Int. Cl. A23L 27/30; A23L 2/60; A61K 8/60; A61K 47/26; A61Q 11/00

(52) U.S. Cl. CPC A23L 27/36; A23L 2/60; A61K 8/602; A61K 47/26; A61Q 11/00

(57) ABSTRACT

The present disclosure relates to the field of natural sweetener compositions useful in the flavor industry. More particularly, it concerns a composition comprising a natural sweetener selected from the group consisting of steviol glycosides, mogrosides, and any mixture thereof, and a solvent mixture comprising A) propylene glycol or glycerol; B) one or more solvents having Hansen Solubility Parameters δₚ and δₕ higher than δₚ and δₕ, respectively, of the solvent chosen as solvent A; and C) one or more solvents having Hansen Solubility Parameters δp and δh lower than δp and δh, respectively, of the solvent chosen as solvent A. The present disclosure further relates to the use of the said composition in consumer products, such as flavored products.

### CROSS REFERENCE TO RELATED APPLICATIONS

This application claims the benefit of priority of European Application No. 22215736.4, filed Dec. 22, 2022, which is hereby incorporated by reference as though set forth herein in its entirety.

### FIELD OF THE DISCLOSURE

The present disclosure relates to the field of natural sweetener compositions useful in the flavor industry. More particularly, it concerns a composition comprising a natural sweetener selected from the group consisting of steviol glycosides, mogrosides, and any mixture thereof, and a solvent mixture comprising A) propylene glycol or glycerol; B) one or more solvents having δₚ and δₕ higher than δₚ and δₕ, respectively, of the solvent chosen as solvent A; and C) one or more solvents having Hansen Solubility Parameters δₚ and δₕ lower than δₚ and δₕ, respectively, of the solvent chosen as solvent A. The present disclosure further relates to the use of the said composition in consumer products, such as flavored products.

### BACKGROUND

Metabolic disorders and related conditions, such as obesity, diabetes, and cardiovascular disease, are major public health concerns throughout the world. And their prevalence is increasing at alarming rates in almost every developed country. Caloric sweeteners, such as sucrose and fructose, are a key contributor to the prevalence of these metabolic disorders and related conditions.

To date, various products have been proposed which seek to address these problems. For instance, artificial high intensity sweeteners have been developed which deliver a sweet taste at very low doses. Of the high intensity sweeteners already present on the market, Sucralose®, Aspartame, Potassium Acesulfame, cyclamate, saccharine are well-known alternatives. However, there is a strong consumer preference for natural or naturally derived products over their artificial counterparts.

Many natural sweeteners, such as steviol glycosides, mogrosides, and the like, exist to meet this consumer need. However, many natural sweeteners are solids that possess a low solubility in the most common flavor solvents, such as water, making them difficult to handle and incorporate into flavored products desired by consumers. While some liquid solutions of natural sweeteners are known, such solutions require heating at elevated temperatures to fully solubilize the natural sweeteners, resulting in solutions that are unstable or stable only for a short time, which presents an obstacle to long-term storage. In other instances, some liquid solutions of natural sweeteners require heating shortly before dilution and use in a final application. The need for thermal treatment presents a clear impact on the cost of production. In yet other instances, some solvents that may be suitable for solubilizing natural sweeteners have the undesirable effect of exhibiting an off taste when used in high concentrations.

Therefore, there is an ongoing need for easy-to-handle natural sweetener solutions that are based on food grade solvents, that are highly concentrated in the sweetener, that are stable (i.e., clear with no precipitation) for long periods of time at room temperature, and that are dilutable in water to reach a target concentration in a final application.

### SUMMARY OF THE DISCLOSURE

The following aspects of the present disclosure seek to address one or more of the problems described hereinabove.

In a first aspect, the present disclosure relates to a composition comprising:
1) a natural sweetener selected from the group consisting of steviol glycosides, mogrosides, and any mixture thereof; and
2) a solvent mixture comprising:
   A) propylene glycol or glycerol;
   B) one or more solvents having Hansen Solubility Parameters δₚ and δₕ higher than δₚ and δₕ, respectively, of the solvent chosen as solvent A; and
C) one or more solvents having Hansen Solubility Parameters δₚ and δₕ lower than δₚ and δₕ, respectively, of the solvent chosen as solvent A.

In a second aspect, the present disclosure relates to a method for preparing the composition described herein.

In a third aspect, the present disclosure relates to a flavored product comprising the composition described herein.

### DETAILED DESCRIPTION

The following detailed description sets forth various aspects and embodiments provided herein. The description is to be read from the perspective of the person of ordinary skill in the relevant art. Therefore, information that is well known to such ordinarily skilled artisans is not necessarily included. It would be apparent to ordinarily skilled artisans that the various aspects and embodiments provided herein may be combined in any manner without departing from the spirit of the disclosure.

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary. Unless defined otherwise, all technical and scientific terms used herein have the same meaning as is commonly understood by one of skill in the art to which this specification pertains.

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

As used herein, “solvate” means a compound formed by the interaction of one or more solvent molecules and one or more compounds described herein. In some embodiments, the solvates are ingestibly acceptable solvates, such as hydrates.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

In the first aspect, the present disclosure relates to a composition comprising:
1) a natural sweetener selected from the group consisting of steviol glycosides, mogrosides, and any mixture thereof; and
2) a solvent mixture comprising:
   A) propylene glycol or glycerol;
   B) one or more solvents having Hansen Solubility Parameters δₚ and δₕ higher than δₚ and δₕ, respectively, of the solvent chosen as solvent A; and
   C) one or more solvents having Hansen Solubility Parameters δₚ and δₕ lower than δₚ and δₕ, respectively, of the solvent chosen as solvent A.

The natural sweetener is selected from the group consisting of steviol glycosides, mogrosides, and any mixture thereof.

As used herein, the term “steviol glycosides” refers to steviol glycosides naturally found in the plants of the species Stevia rebaudiana, Stevia phlebophylla, or Rubus chingii. Such compounds are glycosides of the diterpene, steviol. Some non-limiting examples include stevioside, rebaudiosides, dulcoside A, and the like.

In an embodiment, the natural sweetener comprises one or more steviol glycosides. In some embodiments, the natural sweetener comprises one or more rebaudiosides selected from the group consisting of rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M, and any combinations thereof.

In some embodiments, the natural sweetener comprises one or more mogrosides. Exemplary mogrosides include, but are not limited to mogroside III, mogroside IV, mogroside V, siamenoside, isomogroside V, mogroside IVE, isomogroside V, mogroside IIIE, 11-oxomogroside V, the alpha isomer of isomogroside V, neomogroside, and any combinations thereof. Additional mogroside compounds that may be suitably used as natural sweeteners are described in U.S. Patent Application Publication No. 2017/0119032. In an embodiment, the natural sweetener comprises one or more mogrosides selected from the group consisting of mogroside III, mogroside IV, mogroside V, siamenoside, isomogroside V, mogroside IVE, isomogroside V, mogroside IIIE, 11-oxomogroside V, the alpha isomer of isomogroside V, neomogroside, and any combinations thereof.

It has been surprisingly found that the compositions of the present disclosure may be highly concentrated in the natural sweetener while also being stable (i.e., clear with no precipitation) for long periods of time at ambient temperature or room temperature (RT), typically at a temperature of less than or equal to 25° C. Thus, in some embodiments, the natural sweetener is present in an amount of from 2% to 50%, typically from 4% to 40%, more typically from 10% to 35%, by weight relative to the total weight of the composition. In some embodiments, the natural sweetener is present in an amount of from 2% to 20%, typically from 2% to 19%, more typically from 2% to 18%, by weight relative to the total weight of the composition. In some embodiments, the natural sweetener is present in an amount of from 2% to 15%, typically from 2% to 13%, more typically from 2% to 11%, by weight relative to the total weight of the composition. In some embodiments, the natural sweetener is present in an amount of from 10% to 20%, typically from 14% to 17%, by weight relative to the total weight of the composition. Solutions that are highly concentrated in the natural sweetener while also being stable for long periods of time at ambient temperature offer the advantages of lower cost of use, lower volumes to be transported, as well as reduced impact on taste (i.e., presence of off-notes due to large amounts of solvents), among others.

As used herein, “solubility at 25° C.” or “solubility at RT” is meant to represent the thermodynamic, equilibrium solubility of a natural sweetener at 25° C. or at RT. The ordinarily skilled artisan would understand that the solubility of any solid compound can be significantly increased with increasing temperature of the solvent system. Subsequently, even once the temperature is brought back to a lower temperature, for example to 25° C., the solid compound may remain in solution for a certain amount of time while being supersaturated. However, this is an apparent solubility due to a non-equilibrium, metastable state with limited shelf-life stability, and therefore is distinguished from thermodynamic solubility at equilibrium. Thermodynamic solubility can, for example, be determined with the so-called shake flask method at a controlled temperature. Alternatively, it can be determined by slowly heating the compound and recording the solubilization temperature after complete dissolution.

The solvent mixture of the composition comprises at least three solvents, solvents A), B), and C), the combination of which has been found to be advantageous in solubilizing natural sweeteners, such as those selected from the group consisting of steviol glycosides, mogrosides, and any mixture thereof. Solvent A is propylene glycol or glycerol. In an embodiment, solvent A is propylene glycol. In another embodiment, solvent A is glycerol.

Solvent B is one or more solvents having Hansen Solubility Parameters δₚ and δₕ higher than δₚ and δₕ, respectively, of the solvent chosen as solvent A.

Hansen Solubility Parameters (HSPs) are physicochemical parameters used to estimate the type of interactive forces responsible for compatibility between materials. Hansen solubility parameters may be used to predict if one material will dissolve in another and form a solution. The basis of the HSP is the assumption that the cohesive energy (E) may be divided into three parts corresponding to atomic dispersion (E_{d}), molecular dipolar interactions (Eₚ), and hydrogen-bonding interactions (Eₕ). Similarly, the total solubility parameter can be divided into three components corresponding to the above-mentioned different types of molecular interactions: dispersion (δ_{d}), polar (δₚ), and hydrogen-bonding (δₕ).

A molecule is assigned three Hansen solubility parameters, each generally measured in MPa^{0.5}:
δ_{d}: The energy from dispersion forces between molecules;
δₚ: The energy from dipolar intermolecular force between molecules; and
δₕ: The energy from hydrogen bonds between molecules.

The Hansen solubility parameters can be treated as co-ordinates for a point in three dimensions also known as the Hansen space. The nearer two molecules are in the Hansen space, the more likely they are to dissolve into each other. Further information with respect to Hansen solubility parameters is available at https://www.hansen-solubility.com/.

Hansen Solubility Parameters of exemplary solvents, typically food grade, can be found in the table below. The values are in units of MPa^{0.5} and were calculated with the program HSPiP, which is available at https://www.hansen-solubility.com/.

| Solvent | δ_{d} | δₚ | δₕ |
|---|---|---|---|
| Water | 15.5 | 16 | 42.3 |
| Glycerol | 17.4 | 11.3 | 27.2 |
| Ethanol | 15.8 | 8.8 | 19.4 |
| Propylene glycol | 16.8 | 10.4 | 21.3 |
| 1-Propanol | 16 | 6.8 | 17.4 |
| 2-Propanol | 15.8 | 6.1 | 16.4 |
| Triethyl citrate | 16.5 | 4.9 | 12 |
| Limonene | 17.2 | 1.8 | 4.3 |
| Ethyl lactate | 16 | 7.6 | 12.5 |

In some embodiments, the average Hansen Solubility Parameter δₚ of the solvent mixture does not differ by more than 1 unit, typically 0.5 unit, from δₚ of the natural sweetener.

In some other embodiments, the average Hansen Solubility Parameter δₕ of the solvent mixture will not differ by more than 2 units, typically 1 unit, from on of the natural sweetener.

In an embodiment, solvent A of the solvent mixture is propylene glycol, and solvent B is glycerol or water. In another embodiment, solvent A of the solvent mixture is propylene glycol, and solvent B is glycerol. In yet another embodiment, solvent A of the solvent mixture is propylene glycol and solvent B is water.

In an embodiment, solvent A of the solvent mixture is glycerol and solvent B is water.

Solvent C is one or more solvents having Hansen Solubility Parameters δₚ and δₕ lower than δₚ and δₕ, respectively, of the solvent chosen as solvent A. Exemplary solvents useful as solvent C include, but are not limited to, ethanol, triethyl citrate, triacetin, ethyl lactate, ethyl acetate, propanol, isopropanol, and the like. In an embodiment, solvent C is selected from the group consisting of ethanol, triethyl citrate, triacetin, ethyl lactate, ethyl acetate, propanol, isopropanol, and any mixture thereof.

In an embodiment, solvent A of the solvent mixture is propylene glycol, solvent B is glycerol or water, and solvent C is selected from the group consisting of ethanol, triethyl citrate, triacetin, ethyl lactate, ethyl acetate, propanol, isopropanol, and any mixture thereof.

In an embodiment, solvent A of the solvent mixture is glycerol, solvent B is water, and solvent C is selected from the group consisting of propylene glycol, ethanol, triethyl citrate, triacetin, ethyl lactate, ethyl acetate, propanol, isopropanol, and any mixture thereof.

The amounts of solvents A, B, and C in the compositions of the present disclosure are not particularly limited. However, in suitable embodiments, solvent A of the solvent mixture is present in an amount of from 10% to 75%, typically 30% to 60%, by weight relative to the total weight of the composition.

In an embodiment, solvent B of the solvent mixture is present in an amount of from 10% to 70%, typically 30% to 60%, by weight relative to the total weight of the composition.

In some embodiments, solvent B of the solvent mixture comprises water in an amount of from 0% to 50%, by weight relative to the total weight of the composition.

In an embodiment, solvent C of the solvent mixture is present in an amount of from 1% to 50%, typically 5% to 40%, by weight relative to the total weight of the composition.

The total amount of solvent (solvents A, B, and C) in the compositions of the present disclosure are not particularly limited. However, in some embodiments, the total amount of solvent is from 10% to 98%, typically 50% to 98%, more typically 50% to 95%, by weight relative to the total weight of the composition.

Solvents A, B, and C in the compositions of the present disclosure may be food grade solvents. As used herein, food grade solvents are solvents having a purity, taste properties, color, odor, etc., suitable for use in food.

The composition may further comprise one or more food grade co-solvents. Exemplary solvents belong to the classes of alkylene glycols, alcohols, oils and flavorings. A nonlimiting list of specific suitable food grade co-solvents include butylene glycol, 1,3-propanediol, vegetable oils, olive oil, soy oil, canola oil, cottonseed oil, corn oil, peanut oil, persic oil, sesame oil, fractionated coconut oil, tributyrin, triglycerides, medium chain triglycerides, ethyl levulinate, butyl stearate, diethyl succinate, diethyl malonate, acetic acid, lactic acid, benzyl alcohol, tetrahydrofurfural alcohol, D-limonene, γ-valerolactone, butyrolactone, polyethylene glycol, polypropylene glycol, or combinations thereof.

The composition is transparent and stable for a period of time, typically several hours, more typically several days, even more typically several weeks, at ambient temperature, typically at a temperature of less than or equal to 25° C.

As used herein, the term transparent means that a composition, in the absence of coloring or fluorescent agents, have transmittance values in the visible light (500-800 nm) of 100% at a path length of 1 cm referenced against demineralized water.

As used herein, the term stable means that a composition does not phase separate and remains transparent without detectable signs of turbidity, cloudiness, or precipitates.

In the second aspect, the present disclosure is related to a method for preparing the composition described herein. The method comprises:
a′) mixing:
   A) propylene glycol or glycerol;
   B) one or more solvents having Hansen Solubility Parameters δₚ and δₕ higher than δₚ and δₕ, respectively, of the solvent chosen as solvent A; and
   C) one or more solvents having Hansen Solubility Parameters δₚ and δₕ lower than δₚ and δₕ, respectively, of the solvent chosen as solvent A, to obtain a solvent mixture;
b′) adding a natural sweetener selected from the group consisting of steviol glycosides, mogrosides, and any mixture thereof, to the mixture obtained in step a′; and
c) mixing the combination resulting from step b′, thereby forming the composition.

In one suitable method, solvent A, solvent B (or mix of solvents B) and solvent C (or mix of solvents C) are mixed using a mechanical stirrer to obtain a clear one-phase liquid. The solid natural sweetener in pure form or a blend of solid natural sweeteners is then added to the transparent solvent mixture and mechanically stirred. Mechanical stirring is maintained until the complete solubilization of the sweetener(s) is observed.

In one embodiment, the mixing step c) is carried out at ambient temperature, typically in a temperature range from 15° C. to 25° C.

In another embodiment, the mixing step c) is carried out at a temperature higher than 25° C. Thereby the quantity of added natural sweetener does not exceed the amount which is equivalent to the thermodynamic solubility in the solvent mixture at 25° C.

The features of the composition, including natural sweeteners, solvents A, B, and C, are as described hereinabove and apply mutatis mutandis to the method described herein.

In the third aspect, the present disclosure relates to a flavored product comprising the composition described herein.

The amount of the composition in the flavored product is not particularly limited. In an embodiment, the composition is present in an amount of from 0.001% to 5%, typically 0.01% to 1%, more typically from 0.1% to 0.5%, by weight relative to the total weight of the flavored product.

In an embodiment, flavored product is a food product, a beverage product, an oral care product, or a pharmaceutical product.

In some embodiments, the flavored product further comprises a cooling compound, a cooling-enhancing compound, a sweetener other than steviol glycosides or mogrosides, or a combination thereof.

As used herein, a “cooling compound” refers to a compound that modulates the TRPM8 receptor.

Exemplary cooling compounds include, but are not limited to, compounds selected from the group consisting of: (−)-menthol, N-ethyl-p-menthyl-3-carboxamide, cubebol, isopulegol, 3-(L-menthoxy)propane-1,2-diol, cis/trans-p-menthane-3,8-diol, 3-(L-menthoxy)-2-methylpropane-1,2-diol, 2-[2-(p-menthan-3-yloxy)ethoxy]ethanol, menthoxyethanol, (1R,2R,4R)-1-(2-hydroxy-4-methylcyclohexyl) ethenone, (1R,2R,5R)—N-ethyl-5-methyl-2-(prop-1-en-2-yl)-cyclohexanecarboxamide, N-(3-hydroxy-4-methoxyphenyl)-2-isopropyl-5,5-dimethyl-cyclohexanecarboxamide, N-[4-(cyanomethyl)phenyl]-2-isopropyl-5,5-dimethyl-cyclohexanecarboxamide, N-(2-Hydroxy-2-phenylethyl)-2-isopropyl-5,5-dimethyl-cyclohexane-1-carboxamide, L-menthol lactate, (−)-menthyl ethylene glycol carbonate, (−)-menthone 1,2-glycerol ketal, D/L-menthone 1,2-glycerol ketal, (−)-menthyl 1-propylene glycol carbonate, (−)-menthyl 2-propylene glycol carbonate, D/L-menthyl 1-propylene glycol carbonate, D/L-menthyl 2-propylene glycol carbonate, (1R,2S,5R)—N-(4-methoxyphenyl)-5-methyl-2-(1-methylethyl)cyclohexanecarboxamide, menthyl ethylamido oxalate, (E)-3-benzo[1,3]dioxol-5-yl-N,N-diphenyl-2-propenamide, 3,4-methylenedioxycinnamic acid, N,N-diphenylamide, N,N-dimethyl (−)-menthyl succinimide, (1R,2S,5R)—N-(4-(carbamoylmethyl)phenyl)-menthylcarboxamide, (−)-menthyl pyrrolidone carboxylate, L-phenylephrine p-menthane carboxamide, (1R,2S,5R)—N-(4-(cyanomethyl)-phenyl)menthylcarboxamide, (1R,2S,5R)—N-(2-(pyridin-2-yl)ethyl)menthylcarboxamide, 6-isopropyl-3,9-dimethyl-1,4-dioxaspiro[4.5]decan-2-one, (1R,2R,4S)-dihydroumbellulol, N-ethyl-p-menthane-3-carboxamide, 2-isopropyl-N,2,3-trimethylbutyramide, ethyl 3-(p-menthane-3-carboxamido)acetate, N-ethyl-2,2-diisopropylbutanamide, N-(1,1-dimethyl-2-hydroxyethyl)-2,2-dimethylbutanamide, N-cyclopropyl-5-methyl-2-isopropyl-cyclohexanecarbonecarboxamide, (−)-menthyl acetoacetate, (−)-menthyl succinate, (−)-menthyl(S)-3-hydroxybutyrate, (−)-menthyl glutarate, 1-[2-hydroxyphenyl]-4-[2-nitrophenyl]-1,2,3,6-tetrahydropyrimidine-2-one, di-(−)-menthyl glutarate, N-(2-hydroxyethyl)-2,3-dimethyl-2-isopropylbutanamide, and any combinations thereof.

In some embodiments, the cooling compound may be a cooling compound set forth in WO 2021/074281, such as, for example, (2-(4-phenyl-1H-imidazol-2-yl)piperidin-1-yl)(1,2,3-thiadiazol-5-yl)methanone, (2-(4-phenyl-1H-imidazol-2-yl)piperidin-1-yl)(thietan-3-yl)methanone, (2,2-dimethylcyclopropyl)(2-(4-phenyl-1H-imidazol-2-yl)piperidin-1-yl)methanone, (2-ethoxycyclopropyl)(2-(4-phenyl-1H-imidazol-2-yl)piperidin-1-yl)methanone, (2-methoxycyclopropyl)(2-(4-phenyl-1H-imidazol-2-yl)piperidin-1-yl)methanone, (2-methylcyclopropyl)(2-(4-phenyl-1H-imidazol-2-yl)piperidin-1-yl)methanone, (2S)-2-methyl-1-(2-(5-(p-tolyl)-1H-imidazol-2-yl)piperidin-1-yl) butan-1-one, (5-methyltetrahydrofuran-3-yl)(2-(4-phenyl-1H-imidazol-2-yl)piperidin-1-yl)methanone, (E)-1-(2-(5-(3,4-difluorophenyl)-1H-imidazol-2-yl)piperidin-1-yl)-2-methylbut-2-en-1-one, (E)-2-methyl-1-(2-(5-(p-tolyl)-1H-imidazol-2-yl)piperidin-1-yl) but-2-en-1-one, (E)-2-methyl-1-(2-(5-(p-tolyl)-1H-imidazol-2-yl)piperidin-1-yl) pent-3-en-1-one, (R)-2-(methylthio)-1-((R)-2-(5-(p-tolyl)-1H-imidazol-2-yl)piperidin-1-yl)propan-1-one, (S)-2-(methylthio)-1-((R)-2-(5-(p-tolyl)-1H-imidazol-2-yl)piperidin-1-yl)propan-1-one, (S)-2-(methylthio)-1-((S)-2-(5-(p-tolyl)-1H-imidazol-2-yl)piperidin-1-yl)propan-1-one, (S)-2-methyl-1-((R)-2-(5-(p-tolyl)-1H-imidazol-2-yl)piperidin-1-yl) butan-1-one, (tetrahydrofuran-3-yl)(2-(5-(p-tolyl)-1H-imidazol-2-yl)piperidin-1-yl)methanone, 1-(2-(1H-benzo[d]imidazol-2-yl)piperidin-1-yl)-2-(methylthio)propan-1-one, 1-(2-(4-(1-methyl-1H-pyrazol-4-yl)-1H-imidazol-2-yl)piperidin-1-yl)-2-(methylthio)propan-1-one, 1-(2-(4-(2,2-difluorobenzo[d][1,3]dioxol-5-yl)-1H-imidazol-2-yl)piperidin-1-yl)-2-(methylthio)propan-1 one, 1-(2-(4-(2,3-dihydrobenzofuran-5-yl)-1H-imidazol-2-yl)piperidin-1-yl)-2-(methylthio)propan-1-one, 1-(2-(4-(2,3-dihydrobenzofuran-6-yl)-1H-imidazol-2-yl)piperidin-1-yl)-2-(methylthio)propan-1-one, 1-(2-(4-(2,5-dimethylphenyl)-1H-imidazol-2-yl)piperidin-1-yl)-2-(methylthio)propan-1-one, 1-(2-(4-(2-chlorophenyl)-1H-imidazol-2-yl)piperidin-1-yl)-2-(methylthio)propan-1-one, 1-(2-(4-(2-cyclopropyloxazol-5-yl)-1H-imidazol-2-yl)piperidin-1-yl)-2-(methylthio)propan-1-one, 1-(2-(4-(2-fluorophenyl)-1H-imidazol-2-yl)piperidin-1-yl)-2-(methylthio)propan-1-one, 1-(2-(4-(2-hydroxyphenyl)-1H-imidazol-2-yl)piperidin-1-yl)-2-(methylthio)propan-1-one, 1-(2-(4-(3-((dimethylamino)methyl)phenyl)-1H-imidazol-2-yl)piperidin-1-yl)-2-(methylthio)propan-1-one, 1-(2-(4-(3,5-difluorophenyl)-1H-imidazol-2-yl)piperidin-1-yl)-2-(methylthio)propan-1-one, 1-(2-(4-(3,5-dimethylisoxazol-4-yl)-1H-imidazol-2-yl)piperidin-1-yl)-2-(methylthio)propan-1-one, 1-(2-(4-(3-chloro-4-methylphenyl)-1H-imidazol-2-yl)piperidin-1-yl)-2-(methylthio)propan-1-one, 1-(2-(4-(3-methylfuran-2-yl)-1H-imidazol-2-yl)piperidin-1-yl)-2-(methylthio)propan-1-one, 1-(2-(4-(4-((1,1-dioxidothiomorpholino)methyl)phenyl)-1H-imidazol-2-yl)piperidin-1-yl)-2-(methylthio)propan-1-one, 1-(2-(4-(4-(1-hydroxyethyl)phenyl)-1H-imidazol-2-yl)piperidin-1-yl)-2-(methylthio)propan-1-one, 1-(2-(4-(4-(difluoromethoxy)-3-fluorophenyl)-1H-imidazol-2-yl)piperidin-1-yl)-2-(methylthio)propan-1-one, 1-(2-(4-(4-(difluoromethyl)thiophen-3-yl)-1H-imidazol-2-yl)piperidin-1-yl)-2-(methylthio)propan-1-one, 1-(2-(4-(4-(methylsulfonyl)phenyl)-1H-imidazol-2-yl)piperidin-1-yl)-2-(methylthio)propan-1-one, 1-(2-(4-(4-(tert-butyl)phenyl)-1H-imidazol-2-yl)piperidin-1-yl)-2-(methylthio)propan-1-one, 1-(2-(4-(4-cyclobutylphenyl)-1H-imidazol-2-yl)piperidin-1-yl)-2-(methylthio)propan-1-one, 1-(2-(4-(4-cyclopropyl-2-fluorophenyl)-1H-imidazol-2-yl)piperidin-1-yl)-2-(methylthio)propan-1-one, 1-(2-(4-(4-fluorophenyl)-1H-imidazol-2-yl)piperidin-1-yl)-2-(methylthio)propan-1-one, 1-(2-(4-(4-hydroxyphenyl)-1H-imidazol-2-yl)piperidin-1-yl)-2-(methylthio)propan-1-one, 1-(2-(4-(4-isopropylphenyl)-1H-imidazol-2-yl)piperidin-1-yl)-2-(methylthio)propan-1-one, 1-(2-(4-(4-methyl-3-(trifluoromethyl)phenyl)-1H-imidazol-2-yl)piperidin-1-yl)-2-(methylthio)propan-1-one, 1-(2-(4-(5-((dimethylamino)methyl)thiophen-2-yl)-1H-imidazol-2-yl)piperidin-1-yl)-2-(methylthio)propan-1-one, 1-(2-(4-(5-methylfuran-2-yl)-1H-imidazol-2-yl)piperidin-1-yl)-2-(methylthio)propan-1-one, 1-(2-(4-(5-methylpyridin-3-yl)-1H-imidazol-2-yl)piperidin-1-yl)-2-(methylthio)propan-1-one, 1-(2-(4-(6-methylpyridin-3-yl)-1H-imidazol-2-yl)piperidin-1-yl)-2-(methylthio)propan-1-one, 1-(2-(4-(benzo[b]thiophen-6-yl)-1H-imidazol-2-yl)piperidin-1-yl)-2-(methylthio)propan-1-one, 1-(2-(4-methyl-5-phenyl-1H-imidazol-2-yl)piperidin-1-yl)-2-(methylthio)propan-1-one, 1-(2-(4-phenyl-1H-imidazol-2-yl)piperidin-1-yl)-2-((trifluoromethyl)thio) ethan-1-one, 1-(2-(4-phenyl-1H-imidazol-2-yl)piperidin-1-yl) butan-1-one, 1-(2-(4-phenyl-1H-imidazol-2-yl)piperidin-1-yl) hexa-3,5-dien-1-one, 1-(2-(4-phenyl-1H-imidazol-2-yl)piperidin-1-yl) pent-4-en-1-one, 1-(2-(4-phenyl-1H-imidazol-2-yl)piperidin-1-yl)propan-1-one, 1-(2-(5-(2,3-difluorophenyl)-1H-imidazol-2-yl)piperidin-1-yl)-2-(methylthio)propan-1-one, 1-(2-(5-(2,4-difluorophenyl)-1H-imidazol-2-yl)piperidin-1-yl)-2-(methylthio)propan-1-one, 1-(2-(5-(2,5-difluorophenyl)-1H-imidazol-2-yl)piperidin-1-yl)-2-(methylthio)propan-1-one, 1-(2-(5-(2-fluoro-4-methoxyphenyl)-1H-imidazol-2-yl)piperidin-1-yl)-2-(methylthio)propan-1-one, 1-(2-(5-(2-fluoro-4-methylphenyl)-1H-imidazol-2-yl)piperidin-1-yl)-2-(methylthio)propan-1-one, 1-(2-(5-(2-methoxypyrimidin-5-yl)-1H-imidazol-2-yl)piperidin-1-yl)-2-(methylthio)propan-1-one, 1-(2-(5-(3-(methylamino)phenyl)-1H-imidazol-2-yl)piperidin-1-yl)-2-(methylthio)propan-1-one, 1-(2-(5-(3,4-difluorophenyl)-1H-imidazol-2-yl)piperidin-1-yl)-2-(methylthio)propan-1-one, 1-(2-(5-(3,4-difluorophenyl)-1H-imidazol-2-yl)piperidin-1-yl)-2-methylbut-3-en-1-one, 1-(2-(5-(3,4-dimethoxyphenyl)-1H-imidazol-2-yl)piperidin-1-yl)-2-(methylthio)propan-1-one, 1-(2-(5-(3,4-dimethylphenyl)-1H-imidazol-2-yl)piperidin-1-yl)-2-(methylthio)propan-1-one, 1-(2-(5-(3-fluoro-4-(methoxymethyl)phenyl)-1H-imidazol-2-yl)piperidin-1-yl)-2-(methylthio)propan-1-one, 1-(2-(5-(3-fluoro-4-methylphenyl)-1H-imidazol-2-yl)piperidin-1-yl)-2-(methylthio)propan-1-one, 1-(2-(5-(3-fluorophenyl)-1H-imidazol-2-yl)piperidin-1-yl)-2-(methylthio)propan-1-one, 1-(2-(5-(3-methoxyisothiazol-4-yl)-1H-imidazol-2-yl)piperidin-1-yl)-2-(methylthio)propan-1-one, 1-(2-(5-(3-methoxyphenyl)-1H-imidazol-2-yl)piperidin-1-yl)-2-(methylthio)propan-1-one, 1-(2-(5-(4-((methylamino)methyl)phenyl)-1H-imidazol-2-yl)piperidin-1-yl)-2-(methylthio)propan-1-one, 1-(2-(5-(4-(2-methoxyethyl)phenyl)-1H-imidazol-2-yl)piperidin-1-yl)-2-(methylthio)propan-1-one, 1-(2-(5-(4-(aminomethyl)phenyl)-1H-imidazol-2-yl)piperidin-1-yl)-2-(methylthio)propan-1-one, 1-(2-(5-(4-(dimethylamino)phenyl)-1H-imidazol-2-yl)piperidin-1-yl)-2-(methylthio)propan-1-one, 1-(2-(5-(4-(hydroxymethyl)phenyl)-1H-imidazol-2-yl)piperidin-1-yl)-2-(methylthio)propan-1-one, 1-(2-(5-(4-acetylphenyl)-1H-imidazol-2-yl)piperidin-1-yl)-2-(methylthio)propan-1-one, 1-(2-(5-(4-aminophenyl)-1H-imidazol-2-yl)piperidin-1-yl)-2-(methylthio)propan-1-one, 1-(2-(5-(4-chloro-3-fluorophenyl)-1H-imidazol-2-yl)piperidin-1-yl)-2-(methylthio)propan-1-one, 1-(2-(5-(4-chlorophenyl)-1H-imidazol-2-yl)piperidin-1-yl)-2-(methylthio)propan-1-one, 1-(2-(5-(4-ethylphenyl)-1H-imidazol-2-yl)piperidin-1-yl)-2-(methylthio)propan-1-one, 1-(2-(5-(4-fluorophenyl)-4H-1,2,4-triazol-3-yl)piperidin-1-yl)-2-(methylthio)propan-1-one, 1-(2-(5-(4-methoxy-2-methylphenyl)-1H-imidazol-2-yl)piperidin-1-yl)-2-(methylthio)propan-1-one, 1-(2-(5-(4-methoxyphenyl)-1H-imidazol-2-yl)piperidin-1-yl)-2-(methylthio)propan-1-one, 1-(2-(5-(4-methyl-6-(methylthio)pyridin-3-yl)-1H-imidazol-2-yl)piperidin-1-yl)-2-(methylthio)propan-1-one, 1-(2-(5-(5-(methoxymethyl)thiophen-3-yl)-1H-imidazol-2-yl)piperidin-1-yl)-2-(methylthio)propan-1-one, 1-(2-(5-(6-methoxypyridin-3-yl)-1H-imidazol-2-yl)piperidin-1-yl)-2-(methylthio)propan-1-one, 1-(2-(5-(benzo[d][1,3]dioxol-5-yl)-1H-imidazol-2-yl)piperidin-1-yl)-2-(methylthio)propan-1-one, 1-(2-(5-(p-tolyl)-1H-imidazol-2-yl)piperidin-1-yl)propan-1-one, 1-(2-(5-benzyl-1H-imidazol-2-yl)piperidin-1-yl)-2-(methylthio)propan-1-one, 1-(2-(5-bromo-1H-imidazol-2-yl)piperidin-1-yl)-2-(methylthio)propan-1-one, 2-(2-(1-(2-(methylthio)propanoyl)piperidin-2-yl)-1H-imidazol-5-yl)benzonitrile, 2-(2-(4-phenyl-1H-imidazol-2-yl)piperidine-1-carbonyl)cyclopropane-1-carbonitrile, 2-(5-methylfuran-2-yl)-1-(2-(4-phenyl-1H-imidazol-2-yl)piperidin-1-yl)propan-1-one, 2-(allylthio)-1-(2-(5-(p-tolyl)-1H-imidazol-2-yl)piperidin-1-yl)propan-1-one, 2-(dimethylamino)-1-(2-(4-phenyl-1H-imidazol-2-yl)piperidin-1-yl) butan-1-one, 2-(dimethylamino)-1-(2-(4-phenyl-1H-imidazol-2-yl)piperidin-1-yl)propan-1-one, 2-(ethylthio)-1-(2-(5-(p-tolyl)-1H-imidazol-2-yl)piperidin-1-yl)propan-1-one, 2-(furan-2-yl)-1-(2-(4-phenyl-1H-imidazol-2-yl)piperidin-1-yl) ethan-1-one, 2-(furan-2-yl)-1-(2-(4-phenyl-1H-imidazol-2-yl)piperidin-1-yl)propan-1-one, 2-(furan-3-yl)-1-(2-(4-phenyl-1H-imidazol-2-yl)piperidin-1-yl) ethan-1-one, 2-(furan-3-yl)-1-(2-(5-(p-tolyl)-1H-imidazol-2-yl)piperidin-1-yl)propan-1-one, 2-(methylamino)-N-(4-(2-(1-(2-(methylthio)propanoyl)piperidin-2-yl)-1H-imidazol-5-yl)phenyl) acetamide, 2-(methylsulfinyl)-1-(2-(5-(p-tolyl)-1H-imidazol-2-yl)piperidin-1-yl)propan-1-one, 2-(methylsulfonyl)-1-(2-(4-phenyl-1H-imidazol-2-yl)piperidin-1-yl) ethan-1-one, 2-(methylsulfonyl)-1-(2-(4-phenyl-1H-imidazol-2-yl)piperidin-1-yl)propan-1-one, 2-(methylthio)-1-(2-(2-(p-tolyl)-2H-tetrazol-5-yl)piperidin-1-yl)propan-1-one, 2-(methylthio)-1-(2-(3-phenyl-1,2,4-oxadiazol-5-yl)piperidin-1-yl)propan-1-one, 2-(methylthio)-1-(2-(4-(1-(trifluoromethyl)-1H-pyrazol-4-yl)-1H-imidazol-2-yl)piperidin-1-yl)propan-1-one, 2-(methylthio)-1-(2-(4-(4-(morpholinomethyl)phenyl)-1H-imidazol-2-yl)piperidin-1-yl)propan-1-one, 2-(methylthio)-1-(2-(4-(5-(trifluoromethyl) furan-2-yl)-1H-imidazol-2-yl)piperidin-1-yl)propan-1-one, 2-(methylthio)-1-(2-(4-(5-(trifluoromethyl)thiophen-2-yl)-1H-imidazol-2-yl)piperidin-1-yl)propan-1-one, 2-(methylthio)-1-(2-(4-(5-methylthiophen-3-yl)-1H-imidazol-2-yl)piperidin-1-yl)propan-1-one, 2-(methylthio)-1-(2-(4-(m-tolyl)-1H-imidazol-2-yl)piperidin-1-yl)propan-1-one, 2-(methylthio)-1-(2-(4-(o-tolyl)-1H-imidazol-2-yl)piperidin-1-yl)propan-1-one, 2-(methylthio)-1-(2-(4-(pyridin-3-yl)-1H-imidazol-2-yl)piperidin-1-yl)propan-1-one, 2-(methylthio)-1-(2-(4-(pyridin-4-yl)-1H-imidazol-2-yl)piperidin-1-yl)propan-1-one, 2-(methylthio)-1-(2-(4-(pyrimidin-5-yl)-1H-imidazol-2-yl)piperidin-1-yl)propan-1-one, 2-(methylthio)-1-(2-(4-(quinoxalin-6-yl)-1H-imidazol-2-yl)piperidin-1-yl)propan-1-one, 2-(methylthio)-1-(2-(4-(thiophen-3-yl)-1H-imidazol-2-yl)piperidin-1-yl)propan-1-one, 2-(methylthio)-1-(2-(4-phenyl-1H-imidazol-2-yl)piperidin-1-yl) ethan-1-one, 2-(methylthio)-1-(2-(5-(2-(trifluoromethyl)phenyl)-1H-imidazol-2-yl)piperidin-1-yl)propan-1-one, 2-(methylthio)-1-(2-(5-(2,3,4-trifluorophenyl)-1H-imidazol-2-yl)piperidin-1-yl)propan-1-one, 2-(methylthio)-1-(2-(5-(2,4,5-trifluorophenyl)-1H-imidazol-2-yl)piperidin-1-yl)propan-1-one, 2-(methylthio)-1-(2-(5-(3-(trifluoromethyl)phenyl)-1H-imidazol-2-yl)piperidin-1-yl)propan-1-one, 2-(methylthio)-1-(2-(5-(4-(trifluoromethoxy)phenyl)-1H-imidazol-2-yl)piperidin-1-yl)propan-1-one, 2-(methylthio)-1-(2-(5-(4-(trifluoromethyl)phenyl)-1H-imidazol-2-yl)piperidin-1-yl)propan-1-one, 2-(methylthio)-1-(2-(5-(4-propylphenyl)-1H-imidazol-2-yl)piperidin-1-yl)propan-1-one, 2-(methylthio)-1-(2-(5-(4-vinylphenyl)-1H-imidazol-2-yl)piperidin-1-yl)propan-1-one, 2-(methylthio)-1-(2-(5-(5-methylthiophen-2-yl)-1H-imidazol-2-yl)piperidin-1-yl)propan-1-one, 2-(methylthio)-1-(2-(5-(p-tolyl)-1,3,4-oxadiazol-2-yl)piperidin-1-yl)propan-1-one, 2-(methylthio)-1-(2-(5-(p-tolyl)-1H-imidazol-2-yl) azepan-1-yl)propan-1-one, 2-(methylthio)-1-(2-(5-(p-tolyl)-1H-imidazol-2-yl)piperidin-1-yl)propan-1-one, 2-(methylthio)-1-(2-(5-(p-tolyl)-4H-1,2,4-triazol-3-yl)piperidin-1-yl)propan-1-one, 2-(methylthio)-1-(2-(5-(p-tolyl)isoxazol-3-yl)piperidin-1-yl)propan-1-one, 2-(methylthio)-1-(2-(5-(p-tolyl)oxazol-2-yl)piperidin-1-yl)propan-1-one, 2-(methylthio)-1-(2-(5-(p-tolyl) thiazol-2-yl)piperidin-1-yl)propan-1-one, 2-(methylthio)-1-(2-(5-phenyl-1H-imidazol-2-yl)piperidin-1-yl)propan-1-one, 2-(methylthio)-1-(2-(5-phenyl-1H-imidazol-2-yl) pyrrolidin-1-yl)propan-1-one, 2-(methylthio)-1-(2-(5-phenyl-4H-1,2,4-triazol-3-yl)piperidin-1-yl)propan-1-one, 2-(methylthio)-1-(2-(5-phenyloxazol-2-yl)piperidin-1-yl)propan-1-one, 2-(methylthio)-1-(4-(5-(p-tolyl)-1H-imidazol-2-yl) thiazolidin-3-yl)propan-1-one, 2,2,3-trimethyl-1-(2-(5-(p-tolyl)-1H-imidazol-2-yl)piperidin-1-yl) but-3-en-1-one, 2,2,3-trimethyl-1-(2-(5-(p-tolyl)-1H-imidazol-2-yl)piperidin-1-yl) butan-1-one, 2,2-dimethyl-1-(2-(5-(p-tolyl)-1H-imidazol-2-yl)piperidin-1-yl) but-3-en-1-one, 2,2-dimethyl-1-(2-(5-(p-tolyl)-1H-imidazol-2-yl)piperidin-1-yl) butan-1-one, 2,3-dimethoxy-1-(2-(4-phenyl-1H-imidazol-2-yl)piperidin-1-yl)propan-1-one, 2,3-dimethyl-1-(2-(4-phenyl-1H-imidazol-2-yl)piperidin-1-yl) butan-1-one, 2,5-difluoro-4-(2-(1-(2-(methylthio)propanoyl)piperidin-2-yl)-1H-imidazol-4-yl)benzonitrile, 2-amino-1-(2-(5-(p-tolyl)-1H-imidazol-2-yl)piperidin-1-yl)propan-1-one, 2-cyclobutyl-1-(2-(4-phenyl-1H-imidazol-2-yl)piperidin-1-yl)propan-1-one, 2-cyclopentyl-2-ethoxy-1-(2-(4-phenyl-1H-imidazol-2-yl)piperidin-1-yl) ethan-1-one, 2-cyclopropyl-1-(2-(4-phenyl-1H-imidazol-2-yl)piperidin-1-yl) ethan-1-one, 2-cyclopropyl-1-(2-(4-phenyl-1H-imidazol-2-yl)piperidin-1-yl)propan-1-one, 2-cyclopropyl-2-methoxy-1-(2-(4-phenyl-1H-imidazol-2-yl)piperidin-1-yl) ethan-1-one, 2-ethoxy-1-(2-(5-(3-fluorophenyl)-1H-imidazol-2-yl)piperidin-1-yl)propan-1-one, 2-ethyl-1-(2-(4-phenyl-1H-imidazol-2-yl)piperidin-1-yl) but-2-en-1-one, 2-ethyl-1-(2-(4-phenyl-1H-imidazol-2-yl)piperidin-1-yl) butan-1-one, 2-fluoro-4-(2-(1-(2-(methylthio)propanoyl)piperidin-2-yl)-1H-imidazol-5-yl)benzonitrile, 2-mercapto-1-(2-(5-(p-tolyl)-1H-imidazol-2-yl)piperidin-1-yl)propan-1-one, 2-methoxy-1-(2-(4-phenyl-1H-imidazol-2-yl)piperidin-1-yl) butan-1-one, 2-methoxy-1-(2-(4-phenyl-1H-imidazol-2-yl)piperidin-1-yl)propan-1-one, 2-methoxy-1-(2-(5-(p-tolyl)-1H-imidazol-2-yl)piperidin-1-yl)propan-1-one, 2-methyl-1-(2-(4-phenyl-1H-imidazol-2-yl)piperidin-1-yl) butan-1-one, 2-methyl-1-(2-(4-phenyl-1H-imidazol-2-yl)piperidin-1-yl)propan-1-one, 2-methyl-1-(2-(5-(p-tolyl)-1H-imidazol-2-yl)piperidin-1-yl) but-3-en-1-one, 2-methyl-1-(2-(5-(p-tolyl)-1H-imidazol-2-yl)piperidin-1-yl) butan-1-one, 2-methyl-1-(2-(5-(p-tolyl)-1H-imidazol-2-yl)piperidin-1-yl) butane-1,3-dione, 2-methyl-1-(2-(5-(p-tolyl)-1H-imidazol-2-yl)piperidin-1-yl) pentan-1-one, 2-methyl-1-(2-(5-(p-tolyl)-1H-imidazol-2-yl)piperidin-1-yl) prop-2-en-1-one, 2-methyl-1-(2-(5-(p-tolyl)-1H-imidazol-2-yl)piperidin-1-yl)propan-1-one, 2-methyl-2-(methylthio)-1-(2-(5-(p-tolyl)-1H-imidazol-2-yl)piperidin-1-yl)propan-1-one, 2-methyl-3-(methylthio)-1-(2-(4-phenyl-1H-imidazol-2-yl)piperidin-1-yl)propan-1-one, 2-methylene-1-(2-(5-(p-tolyl)-1H-imidazol-2-yl)piperidin-1-yl) butan-1-one, 3-(2-(1-(2-(methylthio)propanoyl)piperidin-2-yl)-1H-imidazol-4-yl)benzonitrile, 3,3,3-trifluoro-2-methyl-1-(2-(4-phenyl-1H-imidazol-2-yl)piperidin-1-yl)propan-1-one, 3,3,4,4,4-pentafluoro-1-(2-(4-phenyl-1H-imidazol-2-yl)piperidin-1-yl) butan-1-one, 3,3-difluoro-1-(2-(4-phenyl-1H-imidazol-2-yl)piperidin-1-yl) butan-1-one, 3-fluoro-4-(2-(1-(2-(methylthio)propanoyl)piperidin-2-yl)-1H-imidazol-5-yl)benzonitrile, 3-methyl-1-(2-(4-phenyl-1H-imidazol-2-yl)piperidin-1-yl) but-2-en-1-one, 3-methyl-1-(2-(4-phenyl-1H-imidazol-2-yl)piperidin-1-yl) butan-1-one, 4-(2-(1-(2-(methylthio)propanoyl)piperidin-2-yl)-1H-imidazol-4-yl)benzonitrile, 4-(2-(1-(2-(methylthio)propanoyl)piperidin-2-yl)-1H-imidazol-5-yl)benzaldehyde, 4,4,4-trifluoro-2-methyl-1-(2-(4-phenyl-1H-imidazol-2-yl)piperidin-1-yl) butan-1-one, bicyclo[1.1.1]pentan-1-yl(2-(4-phenyl-1H-imidazol-2-yl)piperidin-1-yl)methanone, cyclobutyl(2-(5-(3-fluorophenyl)-1H-imidazol-2-yl)piperidin-1-yl)methanone, cyclopentyl(2-(4-phenyl-1H-imidazol-2-yl)piperidin-1-yl)methanone, cyclopropyl(2-(4-phenyl-1H-imidazol-2-yl)piperidin-1-yl)methanone, methyl 4-(2-(1-(2-(methylthio)propanoyl)piperidin-2-yl)-1H-imidazol-5-yl)benzoate, N-(2-hydroxyethyl)-4-(2-(1-(2-(methylthio)propanoyl)piperidin-2-yl)-1H-imidazol-4-yl)benzenesulfonamide, 2-(methylthio)-1-(2-(5-(3-nitrophenyl)-1H-imidazol-2-yl)piperidin-1-yl)propan-1-one, N-(4-(2-(1-(2-(methylthio)propanoyl)piperidin-2-yl)-1H-imidazol-4-yl)phenyl) methane-sulfonamide, 2-(methylthio)-1-(2-(5-(4-nitrophenyl)-1H-imidazol-2-yl)piperidin-1-yl)propan-1-one, 1-(sec-butylsulfinyl)-2-(5-(p-tolyl)-1H-imidazol-2-yl)piperidine, 1-(sec-butylsulfonyl)-2-(5-(p-tolyl)-1H-imidazol-2-yl)piperidine, 1-(isopropylsulfinyl)-2-(5-(p-tolyl)-1H-imidazol-2-yl)piperidine, 1-(isopropylsulfonyl)-2-(5-(p-tolyl)-1H-imidazol-2-yl)piperidine, 1-((1-(methylthio)ethyl)sulfinyl)-2-(5-(p-tolyl)-1H-imidazol-2-yl)piperidine, 1-((1-(methylthio)ethyl) sulfonyl)-2-(5-(p-tolyl)-1H-imidazol-2-yl)piperidine, 1-((2-(methylthio)propan-2-yl)sulfinyl)-2-(5-(p-tolyl)-1H-imidazol-2-yl)piperidine, 1-((2-(methylthio)propan-2-yl) sulfonyl)-2-(5-(p-tolyl)-1H-imidazol-2-yl)piperidine, 1-(but-3-en-2-ylsulfinyl)-2-(5-(p-tolyl)-1H-imidazol-2-yl)piperidine, 1-(but-3-en-2-ylsulfonyl)-2-(5-(p-tolyl)-1H-imidazol-2-yl)piperidine, 1-(pent-3-en-2-ylsulfinyl)-2-(5-(p-tolyl)-1H-imidazol-2-yl)piperidine, 1-(pent-3-en-2-ylsulfonyl)-2-(5-(p-tolyl)-1H-imidazol-2-yl)piperidine, 1-((2-methylbut-3-en-2-yl)sulfinyl)-2-(5-(p-tolyl)-1H-imidazol-2-yl)piperidine, 1-((2-methylbut-3-en-2-yl) sulfonyl)-2-(5-(p-tolyl)-1H-imidazol-2-yl)piperidine, 1-(2-(5-(4-(hydroxymethyl)-phenyl)-1H-imidazol-2-yl)piperidin-1-yl)-2-methylbutan-1-one, 2-(methylsulfonyl)-1-(2-(5-(p-tolyl)-1H-imidazol-2-yl)piperidin-1-yl)propan-1-one, 1-(4-hydroxy-2-(5-(p-tolyl)-1H-imidazol-2-yl)piperidin-1-yl)-2-(methylthio)propan-1-one, 1-(5-hydroxy-2-(5-(p-tolyl)-1H-imidazol-2-yl)piperidin-1-yl)-2-(methylthio)propan-1-one, 1-(4-hydroxy-2-(5-(p-tolyl)-1H-imidazol-2-yl)piperidin-1-yl)-2-methylbutan-1-one, 1-(5-hydroxy-2-(5-(p-tolyl)-1H-imidazol-2-yl)piperidin-1-yl)-2-methylbutan-1-one, 1-(2-(5-(4-(hydroxymethyl)phenyl)-1H-imidazol-2-yl)piperidin-1-yl)-2-(methylsulfonyl)propan-1-one, 1-(4-hydroxy-2-(5-(p-tolyl)-1H-imidazol-2-yl)piperidin-1-yl)-2-(methylsulfonyl)propan-1-one, 1-(5-hydroxy-2-(5-(p-tolyl)-1H-imidazol-2-yl)piperidin-1-yl)-2-(methylsulfonyl)propan-1-one, 1-(2-hydroxy-6-(5-(p-tolyl)-1H-imidazol-2-yl)piperidin-1-yl)-2-(methylthio)propan-1-one, 1-(2-hydroxy-6-(5-(p-tolyl)-1H-imidazol-2-yl)piperidin-1-yl)-2-methylbutan-1-one, 1-(2-hydroxy-6-(5-(p-tolyl)-1H-imidazol-2-yl)piperidin-1-yl)-2-(methylsulfonyl)propan-1-one, 1-(3-hydroxy-2-(5-(p-tolyl)-1H-imidazol-2-yl)piperidin-1-yl)-2-(methylthio)propan-1-one, 1-(3-hydroxy-2-(5-(p-tolyl)-1H-imidazol-2-yl)piperidin-1-yl)-2-methylbutan-1-one, 1-(3-hydroxy-2-(5-(p-tolyl)-1H-imidazol-2-yl)piperidin-1-yl)-2-(methylsulfonyl)propan-1-one, 1-(2-(5-(4-(hydroxymethyl)phenyl)-1H-imidazol-2-yl)piperidin-1-yl)-2-(methylsulfinyl)-propan-1-one, 1-(4-hydroxy-2-(5-(p-tolyl)-1H-imidazol-2-yl)piperidin-1-yl)-2-(methylsulfinyl)propan-1-one, 1-(5-hydroxy-2-(5-(p-tolyl)-1H-imidazol-2-yl)piperidin-1-yl)-2-(methylsulfinyl)propan-1-one, 1-(2-hydroxy-6-(5-(p-tolyl)-1H-imidazol-2-yl)piperidin-1-yl)-2-(methylsulfinyl)propan-1-one, 1-(3-hydroxy-2-(5-(p-tolyl)-1H-imidazol-2-yl)piperidin-1-yl)-2-(methylsulfinyl)propan-1-one, 1-(2-hydroxy-6-(5-(4-(hydroxymethyl)phenyl)-1H-imidazol-2-yl)piperidin-1-yl)-2-(methylthio)propan-1-one, 1-(5-hydroxy-2-(5-(4-(hydroxymethyl)phenyl)-1H-imidazol-2-yl)piperidin-1-yl)-2-(methylthio)propan-1-one, 1-(4-hydroxy-2-(5-(4-(hydroxymethyl)phenyl)-1H-imidazol-2-yl)piperidin-1-yl)-2-(methylthio)propan-1-one, 1-(3-hydroxy-2-(5-(4-(hydroxymethyl)-phenyl)-1H-imidazol-2-yl)piperidin-1-yl)-2-(methylthio)propan-1-one, 1-(2-hydroxy-6-(5-(4-(hydroxymethyl)phenyl)-1H-imidazol-2-yl)piperidin-1-yl)-2-methylbutan-1-one, 1-(5-hydroxy-2-(5-(4-(hydroxymethyl)phenyl)-1H-imidazol-2-yl)piperidin-1-yl)-2-methylbutan-1-one, 1-(4-hydroxy-2-(5-(4-(hydroxymethyl)phenyl)-1H-imidazol-2-yl)piperidin-1-yl)-2-methylbutan-1-one, 1-(3-hydroxy-2-(5-(4-(hydroxymethyl)phenyl)-1H-imidazol-2-yl)piperidin-1-yl)-2-methylbutan-1-one, 4-(2-(1-(2-methylbutanoyl)piperidin-2-yl)-1H-imidazol-5-yl)benzaldehyde, 4-(2-(1-(2-(methylthio)propanoyl)piperidin-2-yl)-1H-imidazol-5-yl)benzoic acid, 4-(2-(1-(2-methylbutanoyl)piperidin-2-yl)-1H-imidazol-5-yl)benzoic acid, (2,2-dimethylcyclopropyl)(2-(5-(p-tolyl)-1H-imidazol-2-yl)piperidin-1-yl)methanone, 1-(2-(5-bromo-1H-imidazol-2-yl)piperidin-1-yl)-2-methylbutan-1-one, 1-(2-(5-(4-chlorophenyl)-1H-imidazol-2-yl)piperidin-1-yl)-2-methylbutan-1-one, 2-(4-(2-(1-(2-(methylthio)propanoyl)piperidin-2-yl)-1H-imidazol-5-yl)phenyl) acetonitrile, 2-(4-(2-(1-(2-methylbutanoyl)piperidin-2-yl)-1H-imidazol-5-yl)phenyl) acetonitrile, 2-(4-(2-(1-(2-methylbutanoyl)piperidin-2-yl)-1H-imidazol-5-yl)phenyl) acetonitrile, 2,3-dimethyl-1-(2-(5-(p-tolyl)-1H-imidazol-2-yl)piperidin-1-yl) butan-1-one, 2-methyl-1-(2-(5-(o-tolyl)-1H-imidazol-2-yl)piperidin-1-yl) butan-1-one, 2-methyl-1-(2-(5-(m-tolyl)-1H-imidazol-2-yl)piperidin-1-yl) butan-1-one, 1-(2-(5-(4-(hydroxymethyl)phenyl)-1H-imidazol-2-yl)piperidin-1-yl)-2-methylbutan-1-one, 1-(isopropylsulfonyl)-2-(5-(p-tolyl)-1H-imidazol-2-yl)piperidine, 1-(sec-butylsulfonyl)-2-(5-(p-tolyl)-1H-imidazol-2-yl)piperidine, 2-methyl-1-(2-(5-(5-methylthiophen-2-yl)-1H-imidazol-2-yl)piperidin-1-yl) butan-1-one, 2-(methylthio)-1-(2-(5-(p-tolyl)-1H-imidazol-2-yl)piperazin-1-yl)propan-1-one, 2-(methylthio)-1-(2-(4-(p-tolyl)oxazol-2-yl)piperidin-1-yl)propan-1-one, 2-methyl-1-(2-(4-(p-tolyl)oxazol-2-yl)piperidin-1-yl) butan-1-one, derivatives thereof, such as those set forth in WO 2022/170519, or any combination thereof.

In some embodiments, the cooling compound may be 2-methyl-1-(2-(5-(p-tolyl)-1H-imidazol-2-yl)piperidin-1-yl) butan-1-one, 2-(methylthio)-1-(2-(5-(p-tolyl)-1H-imidazol-2-yl)piperidin-1-yl)propan-1-one, 2-methyl-2-(methylthio)-1-(2-(5-(p-tolyl)-1H-imidazol-2-yl)piperidin-1-yl)propan-1-one, 2,2-dimethyl-1-(2-(5-(p-tolyl)-1H-imidazol-2-yl)piperidin-1-yl) but-3-en-1-one, or a combination thereof.

In some embodiments, the cooling compound may be 1-isopropyl-3,3,5,7-tetramethyloctahydrobenzo[c]isoxazole, 5-(4-fluorophenyl)-1,3,3,5,7-pentamethyloctahydrobenzo[c]isoxazole (including (3aR,5R,7S,7aR)-5-(4-fluorophenyl)-1,3,3,5,7-pentamethyloctahydrobenzo[c]isoxazole, (3aR,5R,7R,7aR)-5-(4-fluorophenyl)-1,3,3,5,7-pentamethyloctahydrobenzo[c]isoxazole, (3aR,5R,7S,7aS)-5-(4-fluorophenyl)-1,3,3,5,7-pentamethyloctahydrobenzo[c]isoxazole, or any combination thereof.

As used herein, a “cooling-enhancing compound” refers to a compound capable of enhancing the cooling sensation provided by a flavored product having a cooling compound but may also provide a cooling sensation alone. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. Exemplary cooling-enhancing compounds include, but are not limited to, compounds selected from the group consisting of N-ethyl-N-(thiophen-2-ylmethyl)-2-(p-tolyloxy) acetamide; N-(1H-pyrazol-3-yl)-N-(thiophen-2-ylmethyl)-2-(p-tolyloxy) acetamide; 2-(4-fluorophenoxy)-N-(1H-pyrazol-3-yl)-N-(thiophen-2-ylmethyl) acetamide; 2-(2-hydroxy-4-methylphenoxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiazol-5-ylmethyl)-acetamide; 5-methoxy-N-(1H-pyrazol-3-yl)-N-(thiophen-2-ylmethyl)benzofuran-2-carboxamide; 2-(4-ethylphenoxy)-N-(1H-pyrazol-3-yl)-N-(thiophen-2-ylmethyl) acetamide; (E/Z)-2-methyl-2-butenal; (E/Z)-2-isopropyl-5-methyl-2-hexenal; derivatives thereof, such as those set forth in WO 2022/170519, and any combinations thereof.

The flavored product may further comprise a sweetener other than steviol glycosides or mogrosides. The sweetener may comprise a low-calorie, zero-calorie, or high-intensity sweetener. The term “high-intensity sweetener” refers to sweeteners that are sweeter than sucrose, such as at least 10 times, or at least 20 times, or at least 50 times, or at least 100 times, or at least 250 times, or at least 500 times, or at least 750 times, or at least 1000 times, or at least 2500 times, or at least 5000 times, or at least 7500 times, or at least 10000 times, sweeter than sucrose. In this context, to say that a sweetener is x times sweeter than sucrose means that a concentration of 1/x by weight relative to sucrose in aqueous solution of said sweetener yields an equivalent sweetness as sucrose.

Examples of suitable sweeteners are set forth in greater detail below. In some embodiments, the sweetener is sucralose, which refers to 1,6-dichloro-1,6-dideoxy-B-D-fructofuranosyl-4-chloro-4-deoxy-α-D-galactopyranoside. In some embodiments, the sweetener is sodium saccharin. The sodium saccharin can be present in any suitable concentration. In some embodiments, the concentration of sodium saccharin ranges from 10 ppm to 10000 ppm, or from 50 ppm to 5000 ppm, or from 100 ppm to 3000 ppm.

In some embodiments, the sweetener may comprise caloric sugar, such as sucrose, glucose, fructose (e.g., in the form of high-fructose corn syrup), or any combination thereof. In some embodiments, the sweetener comprises one or more high-intensity artificial sweeteners, such as acefulfame potassium, aspartame, cyclamate, neotame, and the like. When any such artificial high-intensity sweeteners are present, their concentration would be about the same as those set forth above for sucralose. In some other embodiments, the sweetener comprises one or more low-calorie carbohydrates or sugar alcohols, such as allulose, xylitol, erythritol, and the like.

Various other sweeteners may also be included in the flavored product of the present disclosure. Non-limiting examples include D-psicose, L-ribose, D-tagatose, L-glucose, L-fucose, L-arbinose, D-turanose, D-leucrose, isomalt, lactitol, mannitol, sorbitol, maltodextrin, saccharin, alitame, cyclamic acid, tagatose, maltose, galactose, mannose, lactose, D-tryptophan, glycine, maltitol, lactitol, isomalt, hydrogenated starch hydrolyzate (HSH), chemically modified mogrosides (such as glucosylated mogrosides), carrelame and other guanidine-based sweeteners, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In a particular embodiment, the flavored product comprises a sweetener selected from the group consisting of sucrose, fructose, glucose, erythritol, sucralose, xylitol, and any combinations thereof.

In some embodiments, the flavored product may comprise 4-amino-5,6-dimethylthieno[2,3-d]pyrimidin-2 (1H)-one, or any suitable salt thereof. In some embodiments, the flavored product disclosed herein may comprise the hydrochloride salt of 4-amino-5,6-dimethylthieno[2,3-d]pyrimidin-2 (1H)-one.

In some embodiments of any of the foregoing embodiments, the flavored product may comprise a lactone. Any suitable lactone or combination of lactones can be used. In some embodiments, the lactone comprises a lactone ring having from 3 to 8 members in the ring, such as a 3-membered lactone ring, a 4-membered lactone ring, a 5-membered lactone ring, a 6-membered lactone ring, a 7-membered lactone ring, or an 8-membered lactone ring.

In some embodiments, the lactone comprises a 5-membered lactone ring with or without an unsaturated bond (excluding the unsaturation in the oxo substituent on the ring), such as a carbon-carbon double bond. In some embodiments, the lactone comprises a 5-membered lactone ring including at least one unsaturated bond (for example, one carbon-carbon double bond). Non-limiting examples of lactones comprising a 5-membered lactone ring having at least one unsaturated bond include angelica lactone alpha, angelica lactone beta, mint lactone, 5,5-dimethyl-2 (5H)-furanone, 5-ethyl-2 (5H)-furanone, 5-pentyl-2 (5H)-furanone, 5-hexyl-2 (5H)-furanone, 5-pentyl-2 (3H)-furanone, 4-methyl-3-pentyl-2 (5H)-furanone, and 2 (5H)-furanone. In some embodiments, the lactone comprises a 5-membered lactone ring without any unsaturated bonds. Non-limiting examples of such lactones include butyrolactone gamma, valerolactone gamma, hexalactone gamma, heptalactone gamma, octalactone gamma, undecalactone gamma, decalactone gamma, dihydrojasmone lactone, and dihydromintlactone (3,6-dimethyl-hexahydro-2 (3H)-benzofuranone). In some embodiments, the lactone is dihydromintlactone.

In some embodiments, the lactone comprises a 6-membered lactone ring with or without an unsaturated bond. In some embodiments, the lactone comprises a 6-membered lactone ring including at least one unsaturated bond. Non-limiting examples of lactones comprising a 6-membered lactone ring having at least one unsaturated bond include δ-2-decenolactone, coumarin, and 6-methylcoumarin. In some other embodiments, the lactone includes a 6-membered lactone ring without any unsaturated bonds. Non-limiting examples of lactones including a 6-membered lactone ring without any unsaturated bonds include decalactone delta, dodecalactone delta, and cyclohexyl lactone.

In certain embodiments, the flavored product further comprises one or more food additives, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example natural flavor, natural flavor with other natural flavors (natural flavor WONF), natural and artificial flavors, artificial flavor; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

In some further embodiments, the flavored product disclosed herein comprises one or more other umami or kokumi enhancing compounds. Such umami enhancing compounds include, but are not limited to, naturally derived compounds, such as ericamide, or synthetic compounds, such as any compounds set forth in U.S. Pat. Nos. 8,735,081; 8,124,121; and 8,968,708.

In some further embodiments, the flavored product disclosed herein may comprise one or more bitterness blocking compounds. Such bitterness blocking compounds include, but are not limited to, naturally derived compounds, such as menthol or analogs thereof, or synthetic compounds, such as any compounds set forth in U.S. Pat. Nos. 8,076,491; 8,445,692; and 9,247,759.

In some further embodiments, the flavored product disclosed herein may comprise one or more mouthfeel modifying compounds. Such mouthfeel modifying compounds include, but are not limited to, tannins, cellulosic materials, bamboo powder, and the like.

In some further embodiments, the flavored product disclosed herein may comprise one or more flavor masking compounds. Such flavor masking compounds include, but are not limited to, cellulosic materials, materials extracted from fungus, materials extracted from plants, citric acid, carbonic acid (or carbonates), and the like.

In certain embodiments, the flavored product disclosed herein may comprise cyclosal. When cyclosal is present, it is present at a relative concentration similar to that of the lactones described herein.

In some embodiments, the flavored product comprises one or more flavorings, or any other additives described in H. Mitchell, Sweeteners and Sugar Alternatives in Food Technology, Blackwell Publishing Ltd, 2006. As used herein, the term “flavorings” includes those flavors known to those of ordinary skill in the art, such as natural and artificial flavors. These flavorings may be chosen from synthetic flavor oils and flavoring aromatics or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Further useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with cooling agents.

Flavors may also provide breath freshening properties, particularly the mint flavors when used in combination with cooling agents. These flavorings may be used in liquid or solid form and may be used individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. Generally, any flavoring or food additive such as those described in Chemicals Used in Food Processing, publication 1274, pages 63-258, by the National Academy of Sciences, may be used.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavorings are merely exemplary and are not meant to limit either the term “flavoring” or the scope of the disclosure generally.

The flavored product can be any flavored product according to the aspects and embodiments set forth below. In some embodiments, the flavored product is a beverage product, such as enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some other embodiments, the flavored product is a food product, such as a packaged meal, canned foods, meal replacement products, and the like. In some embodiments, the flavored product is an oral care product, such as a toothpaste, a mouthwash, or a teeth-whitening composition, such as a composition disclosed in U.S. Patent Application Pub. No. 2019/0167545, which is incorporated herein by reference.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

The flavored product can be in any suitable physical form. For example, in some embodiments, the flavored product can be in the form of a solid, such as a powder. In a further such embodiment, the flavored product is a toothpowder, such as a toothpowder for whitening teeth, removing stains (e.g., tobacco stains), and the like. In some other embodiments, the flavored product can be a paste or a slurry, such as a toothpaste. In some other embodiments, the flavored product can be in the form of a cream or gel, such as a tooth gel, a skin cream or tooth cream. In some other embodiments, the flavored product can be a liquid suspension or solution, for example, with an aqueous carrier. The flavored product can, in some embodiments, be in the form of an emulsion, such as a microemulsion or nanoemulsion, where a surfactant, emulsifier, and the like can be present. In some embodiments, the flavored product is a personal care, such as a product for application onto skin, face, lips, gums, mouth, and the like. Such personal products can be for any suitable use, such as skin whitening, teeth whitening, and the like.

In some embodiments, the flavored product is an oral care product. By “oral care product” is meant a personal care product or other product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care composition may be in various forms including toothpaste, toothpaste powder, dentifrice, tooth gel, subgingival gel, mouthrinse, mousse, foam, mouthspray, lozenge, chewable tablet, chewing gum or denture product. The oral care composition may also be incorporated onto strips or films for direct application or attachment to oral surfaces.

In some embodiments, the flavored product is a pharmaceutical product, such as a product designed to carry or deliver certain active pharmaceutical ingredients (APIs). Such APls can be over-the-counter (OTC) drugs, such as acetaminophen and other OTC cough and cold medications. In some embodiments, the API is a prescription-based drug.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or iced tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof. Further examples include, but are not limited to packaged/industrial bread, unpackaged/artisanal bread, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, and hot cereals.

The Ice Cream category generally refers to frozen dessert containing cream and sugar and flavoring. Examples of ice cream include, but are not limited to, impulse ice cream; take-home ice cream; frozen yoghurt and artisanal ice cream; soy, oat, bean (e.g., red bean and mung bean), and rice-based ice creams. Further examples of ice cream include, but are not limited to, single portion dairy ice cream, single portion water ice cream, multi-pack dairy ice cream, multi-pack water ice cream, take-home dairy ice cream, ice cream desserts, bulk ice cream, and take-home water ice cream.

The Confectionery category generally refers to edible product that is sweet to the taste. Examples of confectionery include, but are not limited to candies, gelatins, chocolate confectionery, sugar confectionery, gum, and the likes and any combination products. Further examples include, but are not limited to, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, and other sugar confectionery.

The Meal Replacement category generally refers to any food intended to replace the normal meals, particularly for people having health or fitness concerns. Examples of meal replacement include, but are not limited to, slimming products and convalescence drinks, and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to, canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy-based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to, milk- or soybean-based formula; and prepared, dried and other baby food.

The Spreads category includes, but is not limited to jams and preserves, honey, chocolate spreads, nut-based spreads, and yeast-based spreads.

The Dairy Product category generally refers to edible product produced from mammal's milk. Examples of dairy products include, but are not limited to drinking milk products, cheese, yoghurt and sour milk drinks, and other dairy products.

Further examples of dairy products include, but are not limited to, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, fermented dairy drinks, coffee whiteners, powder milk, flavored powder milk drinks, cream, processed cheese, spreadable processed cheese, unspreadable processed cheese, unprocessed cheese, spreadable unprocessed cheese, hard cheese, packaged hard cheese, unpackaged hard cheese, plain/natural yoghurt, flavored yoghurt, fruited yoghurt, probiotic yoghurt, drinking yoghurt, regular drinking yoghurt, and probiotic drinking yoghurt.

In some embodiments, the flavored product comprises one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads.

Some embodiments provide a chewable flavored product that may or may not be intended to be swallowed. In some embodiments, the chewable flavored product may be gum, chewing gum, sugarized gum, sugar-free gum, functional gum, bubble gum including compounds as disclosed and described herein, individually or in combination.

The compositions, methods, and products according to the present disclosure are further illustrated by the following non-limiting examples.

### EXAMPLES

Unless otherwise stated, the inventive compositions of the examples were prepared according to the following general procedure. Solvent A, Solvent B (or mix of solvents B) and solvent C (or mix of solvents C) were mixed using a mechanical stirrer at room temperature (22° C.) to obtain a clear one-phase liquid. A solid natural sweetener in pure form or a blend of solid natural sweeteners was then added to the transparent solvent mixture and mechanically stirred. Mechanical stirring was maintained until the complete solubilization of the sweetener(s) at room temperature was observed. Comparative compositions were prepared analogously. The tables in the following examples summarize the components used in the inventive and comparative compositions.

### Example 1

Comparative and inventive compositions were prepared according to Table 1 below. The solubilization of the sweetener was assessed visually as a function of time. The stability of the concentrated solution (transparency of the solution) was also assessed visually at room temperature (22° C.) as a function of time.

TABLE 1

| | Comparative | Comparative | |
|---|---|---|---|
| | sample C1 | sample C2 | Sample 1 |
| | % wt | % wt | % wt |
| Solvent A: Propylene | 48.1 | 47.7 | 45.3 |
| glycol¹⁾ | | | |
| Solvent B: Glycerol²⁾ | 48.1 | 47.7 | 45.3 |
| Solvent C: Ethanol³⁾ | | | 4.8 |
| Sweetener: Reb A | 3.8 | 4.6 | 4.6 |
| (99.5%)⁴⁾ | | | |
| Solution aspect | Clear | Turbid | Clear |
| Increase solubility factor | | | 1.2 |

¹⁾Propylene glycol (origin: Carlo Erba)
²⁾Glycerol 99.5% (origin: Alfa Aesar)
³⁾Ethanol absolute (origin: Carlo Erba)
⁴⁾Steviol glycoside Rebaudioside A 99.5% (origin: Firmenich)

In the examples above, a binary mix of solvents containing propylene glycol and glycerol in a 50/50 weight ratio was prepared. Dispersing 4.6% wt. of Reb A (99.5%) in the binary mix resulted in a non-complete solubilization of active and a cloudy solution after 2 days at room temperature (22° C.), while 3.8% wt. of Reb A (99.5%) was soluble. Adding 5% wt. of a 3^{rd} solvent (ethanol) to the binary mix resulted in the solubilization of the same amount of Reb A (99.5%). Sample 1 according to the invention was clear and stable over several weeks at room temperature. Adding a 3^{rd} solvent to the binary mix of 50/50 propylene glycol/glycerol, where the 3^{rd} solvent has Hansen Solubility Parameters δₚ and δₕ strictly lower than solvent A (propylene glycol) and solvent B (glycerol), provided an increase in the solubility of Reb A (99.5%) by a factor of about 1.2.

### Example 2

Comparative and inventive compositions were prepared according to Table 2 below.

TABLE 2

| | Comparative | Comparative | |
|---|---|---|---|
| | sample C3 | sample C4 | Sample 2 |
| | % wt | % wt | % wt |
| Solvent A: Propylene | 32.1 | 31.8 | 28.6 |
| glycol¹) | | | |
| Solvent B: Glycerol²⁾ | 64.3 | 63.6 | 57.2 |
| Solvent C: Ethanol³⁾ | | | 9.6 |
| Sweetener: Reb A | 2.6 | 4.6 | 4.6 |
| (99.5%)⁴⁾ | | | |
| Solution aspect | Clear | Turbid | Clear |
| Increase solubility factor | | | 1.7 |

¹⁾Propylene glycol (origin: Carlo Erba)
²⁾Glycerol 99.5% (origin: Alfa Aesar)
³⁾Ethanol absolute (origin: Carlo Erba)
⁴⁾Steviol glycoside Rebaudioside A 99.5% (origin: Firmenich)

In the examples above, a binary mix of solvents containing propylene glycol and glycerol in 33/66 weight ratio was prepared. Dispersing 4.6% wt. of Reb A (99.5%) in the binary mix resulted in a non-complete solubilization of active and a cloudy solution after 2 days at room temperature (22° C.), while 2.6% wt. of Reb A (99.5%) was soluble. Adding 10% wt. of a 3^{rd} solvent (ethanol) to the binary mix resulted in the solubilization of the same amount of Reb A (99.5%). Sample 2 according to the invention was clear and stable over several weeks at room temperature. Adding the 3^{rd} solvent to the binary mix (33/66 propylene glycol/glycerol), where the 3^{rd} solvent has Hansen Solubility Parameters δₚ and δₕ strictly lower than solvent A (propylene glycol) and solvent B (glycerol), provided an increase in the solubility of Reb A (99.5%) by a factor of 1.7.

### Example 3

Comparative and inventive compositions were prepared according to Table 3 below.

TABLE 3

| | Comparative | Comparative | |
|---|---|---|---|
| | sample C5 | sample C6 | Sample 3 |
| | % wt | % wt | % wt |
| Solvent A: Propylene | 47.45 | 45.5 | 43.2 |
| glycol¹⁾ | | | |
| Solvent B: Glycerol²⁾ | 47.45 | 45.5 | 43.2 |
| Solvent C: Ethanol³⁾ | | | 4.6 |
| Sweetener: Reb A | 5.1 | 9 | 9 |
| (80%)⁴⁾ | | | |
| Solution aspect | Clear | Turbid | Clear |
| Increase solubility factor | | | 1.7 |

¹⁾Propylene glycol (origin: Carlo Erba)
²⁾Glycerol 99.5% (origin: Alfa Aesar)
³⁾Ethanol absolute (origin: Carlo Erba)
⁴⁾Stevia Extract SG95 Rebaudioside A 80% (origin: Firmenich)

In the examples above, a binary mix of solvents containing propylene glycol and glycerol in a 50/50 weight ratio was prepared. Dispersing 9% wt. of Reb A (80%) in the binary mix resulted in a non-complete solubilization of active and a cloudy solution after 1 day at room temperature (22° C.), while 5.1% wt. of Reb A (80%) was soluble. Adding 5% wt. of a 3^{rd} solvent (ethanol) to the binary mix resulted in the solubilization of the same amount of RebA (80%). Sample 3 was clear and stable over several weeks at room temperature. Adding the 3^{rd} solvent to the binary mix (50/50 propylene glycol/glycerol), where the 3^{rd} solvent has Hansen Solubility Parameters δₚ and δₕ strictly lower than solvent A (propylene glycol) and solvent B (glycerol), provided an increase in the solubility of Reb A (80%) by a factor of 1.7.

### Example 4

Comparative and inventive compositions were prepared according to Table 4 below.

TABLE 4

| | Comparative | Comparative | | Sample |
|---|---|---|---|---|
| | sample C7 | sample C8 | Sample 4 | 5 |
| | % wt | % wt | % wt | % wt |
| Solvent A: Propylene | 43.5 | 42.6 | 39.65 | 40.47 |
| glycol¹⁾ | | | | |
| Solvent B: Glycerol²⁾ | 43.5 | 42.6 | 39.65 | 40.47 |
| Solvent C: Ethanol³⁾ | | | 4.2 | |
| Solvent C: | | | | 4.26 |
| Triethylcitrate⁴⁾ | | | | |
| Sweetener: | 13 | 14.8 | 16.5 | 14.8 |
| Reb M (80%)⁵⁾ | | | | |
| Solution aspect | Clear | Turbid | Clear | Clear |
| Increase solubility | | | 1.28 | 1.14 |
| factor | | | | |

¹⁾Propylene glycol (origin: Carlo Erba)
²⁾Glycerol 99.5% (origin: Alfa Aesar)
³⁾Ethanol absolute (origin: Carlo Erba)
⁴⁾Triethylcitrate (origin: Firmenich)
⁵⁾Steviol glycosides Reb M 80% (origin: Firmenich)

In the examples above, a binary mix of solvents containing propylene glycol and glycerol in a 50/50 weight ratio was prepared. Dispersing 14.8% wt. of Reb M (80%) in the binary mix resulted in a non-complete solubilization of active and a cloudy solution after 1 day at room temperature (22° C.), while 13% wt. of Reb M (80%) was soluble. Adding 5% wt. of a 3^{rd} solvent (triethylcitrate) to the binary mix resulted in the solubilization of the same amount of Reb M (80%). Adding 5% wt. of ethanol as a 3^{rd} solvent to the binary mix resulted in the solubilization of an even much higher concentration of Reb M (80%), up to 16.5% wt. Samples 4 and 5 were clear and stable over several weeks at room temperature. Adding a 3^{rd} solvent to the binary mix (50/50 propylene glycol/glycerol), where the 3^{rd} solvent has Hansen Solubility Parameters δₚ and δₕ strictly lower than solvent A (propylene glycol) and solvent B (glycerol), provided an increase in the solubility of Reb M (80%) by a factor of 1.28 when the 3^{rd} solvent was ethanol and 1.14 when the 3^{rd} solvent was triethylcitrate.

### Example 5

Comparative and inventive compositions were prepared according to Table 5 below.

TABLE 5

| | Comparative | Comparative | |
|---|---|---|---|
| | Sample C9 | Sample C10 | Sample 6 |
| | % wt | % wt | % wt |
| Solvent A: Propylene glycol¹⁾ | 72 | 71.2 | 64.08 |
| Solvent B: Water²⁾ | 18 | 17.8 | 16.02 |
| Solvent C: Triethylcitrate³⁾ | | | 8.9 |
| Sweetener: Reb A (99.5%)⁴⁾ | 10.0 | 11.0 | 11.0 |
| Solution aspect | Clear | Turbid | Clear |
| Increase solubility factor | | | 1.1 |

¹⁾Propylene glycol (origin: Carlo Erba)
²⁾DI water
³⁾Triethylcitrate (origin: Firmenich)
⁴⁾Steviol glycoside Rebaudioside A 99.5% (origin: Firmenich)

In the examples above, a binary mix of solvents containing propylene glycol and water in a weight ratio of 80/20 was prepared. Dispersing 11% wt. of Reb A (99.5%) in the binary mix resulted in a non-complete solubilization of active and a cloudy solution after 2 days at room temperature (22° C.), while 10% wt. of Reb A (99.5%) was soluble. Adding 10% wt. of a 3^{rd} solvent (triethylcitrate) to the binary mix resulted in the solubilization of the same amount of Reb A (99.5%). Sample 6 was clear and stable over several weeks at room temperature. Adding the 3^{rd} solvent to the binary mix (80/20 propylene glycol/water), where the 3^{rd} solvent has Hansen Solubility Parameters δₚ and δₕ strictly lower than solvent A (propylene glycol) and solvent B (water), provided an increase in the solubility of Reb A (99.5%) by a factor of 1.1.

### Example 6

Comparative and inventive compositions were prepared according to Table 6 below.

TABLE 6

| | Comparative Sample | |
|---|---|---|
| | C11 | Sample 7 |
| | % wt | % wt |
| Solvent A: Glycerol¹⁾ | 78.4 | 70.56 |
| Solvent B: Water²⁾ | 19.6 | 17.64 |
| Solvent C: Isopropanol³⁾ | | 9.8 |
| Sweetener: Reb A (99.5%)⁴⁾ | 2.0 | 2.0 |
| Solution aspect | Turbid | Clear |
| Increase solubility factor | | >1 |

¹⁾Glycerol 99.5% (origin: Alfa Aesar)
²⁾DI water
³⁾Propan-2-ol (origin: Carlo Erba)
⁴⁾Steviol glycoside Rebaudioside A 99.5% (origin: Firmenich)

In the examples above, a binary mix of solvent containing glycerol and water in an 80/20 weight ratio was prepared. Dispersing 2% wt. of Reb A (99.5%) in the binary mix resulted in a non-complete solubilization of active and a cloudy solution after 1 day at room temperature (22° C.). Adding 10% wt. of a 3^{rd} solvent (isopropanol) to the binary mix resulted in the solubilization of the same amount of Reb A (99.5%). Example 7 was clear and stable over several weeks at room temperature. Adding a 3^{rd} solvent to the binary mix (80/20 glycerol/water) where the 3^{rd} solvent has Hansen Solubility Parameters δₚ and δₕ strictly lower than solvent A (glycerol) and solvent B (water), provided an increase in the solubility of Reb A (99.5%) by a factor of greater than 1.

### Example 7

Comparative and inventive compositions were prepared according to Table 7 below.

TABLE 7

| | Comparative | Comparative | |
|---|---|---|---|
| | sample C12 | sample C13 | Sample 8 |
| | % wt | % wt | % wt |
| Solvent A: Glycerol¹⁾ | 79.2 | | 28.2 |
| Solvent B: Water²⁾ | 19.8 | 19.4 | 17.6 |
| Solvent C: Ethanol³⁾ | | 77.6 | 42.2 |
| Sweetener: Reb A (99.5%)⁴⁾ | 1.0 | 3.0 | 12.0 |
| Solution aspect | Turbid | Turbid | Clear |
| Increase solubility factor | | | >4 |

¹⁾Glycerol 99.5% (origin: Alfa Aesar)
²⁾DI water
³⁾Ethanol absolute (origin: Carlo Erba)
⁴⁾Steviol glycoside Rebaudioside A 99.5% (origin: Firmenich)

In the examples above, a first binary mix of solvent containing glycerol and water in an 80/20 weight ratio was prepared. Separately, a binary mix of solvents containing ethanol and water in an 80/20 weight ratio was also prepared. Dispersing 1% wt. of Reb A (99.5%) in the binary mix glycerol/water 80/20 resulted in a non-complete solubilization of active and a cloudy solution after 1 day at room temperature (22° C.). Dispersing 3% wt. of Reb A (99.5%) in the binary mix ethanol/water 80/20 resulted in a non-complete solubilization of active and a cloudy solution after 1 day at room temperature (22° C.). Replacing 48% wt. of glycerol by a 3^{rd} solvent (ethanol) into the first binary mix resulted in the solubilization of 12% of Reb A (99.5%). Sample 8 was clear and stable over several weeks at room temperature. Adding a 3^{rd} solvent to the binary mix (80/20 glycerol/water) where the 3^{rd} solvent has Hansen Solubility Parameters δₚ and δₕ strictly lower than solvent A (glycerol) and solvent B (water), provided an increase in the solubility of Reb A (99.5%) by a factor of greater than 4.

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

1. A composition comprising:
1) a natural sweetener selected from the group consisting of steviol glycosides, mogrosides, and any mixture thereof; and
2) a solvent mixture comprising:
A) propylene glycol or glycerol;
B) one or more solvents having Hansen Solubility Parameters δ_{P} and δₕ higher than δₚ and δₕ, respectively, of the solvent chosen as solvent A; and
C) one or more solvents having Hansen Solubility Parameters δₚ and δₕ lower than δₚ and δₕ, respectively, of the solvent chosen as solvent A.

2. The composition according to claim 1, wherein the natural sweetener is present in an amount of from 2% to 50%, by weight relative to the total weight of the composition.

3. The composition according to claim 1, wherein the natural sweetener comprises one or more steviol glycosides.

4. The composition according to claim 1, wherein the natural sweetener comprises one or more mogrosides selected from the group consisting of mogroside III, mogroside IV, mogroside V, siamenoside, isomogroside V, mogroside IVE, isomogroside V, mogroside IIIE, 11-oxomogroside V, the alpha isomer of isomogroside V, neomogroside, and any combinations thereof.

5. The composition according to claim 1, wherein solvent A of the solvent mixture is propylene glycol, solvent B is glycerol or water, and solvent C is selected from the group consisting of ethanol, triethyl citrate, triacetin, ethyl lactate, ethyl acetate, propanol, isopropanol, and any mixture thereof.

6. The composition according to claim 1, wherein solvent A of the solvent mixture is glycerol, solvent B is water, and solvent C is selected from the group consisting of propylene glycol, ethanol, triethyl citrate, triacetin, ethyl lactate, ethyl acetate, propanol, isopropanol, and any mixture thereof.

7. The composition according to claim 1, wherein solvent A of the solvent mixture is present in an amount of from 10% to 75%, typically 30% to 60%, by weight relative to the total weight of the composition.

8. The composition according to claim 1, wherein solvent B of the solvent mixture is present in an amount of from 10% to 70% by weight relative to the total weight of the composition.

9. The composition according to claim 1, wherein solvent C of the solvent mixture is present in an amount of from 1% to 50% by weight relative to the total weight of the composition.

10. The composition according to claim 1, wherein the composition is transparent and stable for a period of time at ambient temperature.

11. A method for preparing the composition according to claim 1, the method comprising:
a′) mixing:
A) propylene glycol or glycerol;
B) one or more solvents having Hansen Solubility Parameters δₚ and δₕ higher than δₚ and δₕ, respectively, of the solvent chosen as solvent A; and
C) one or more solvents having Hansen Solubility Parameters δₚ and δₕ lower than δₚ and δₕ, respectively, of the solvent chosen as solvent A, to obtain a solvent mixture;
b′) adding a natural sweetener selected from the group consisting of steviol glycosides, mogrosides, and any mixture thereof, to the mixture obtained in step a′; and
c) mixing the combination resulting from step b′, thereby forming the composition.

12. A flavored product comprising the composition according to claim 1.

13. The flavored product according to claim 12, wherein the composition is present in an amount of from 0.001% to 5%, by weight relative to the total weight of the flavored product.

14. The flavored product according to claim 12, wherein the flavored product is a food product, a beverage product, an oral care product, or a pharmaceutical product.

15. The flavored product according to claim 12, wherein the flavored product further comprises a cooling compound, a cooling-enhancing compound, a sweetener other than steviol glycosides or mogrosides, or a combination thereof.

16. The composition according to claim 3, wherein the natural sweetener comprises one or more rebaudiosides selected from the group consisting of rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M, and any combinations thereof.

17. The composition according to claim 7, wherein solvent A of the solvent mixture is present in an amount of from 30% to 60% by weight relative to the total weight of the composition.

18. The composition according to claim 8, wherein solvent B of the solvent mixture is present in an amount of from 30% to 60% by weight relative to the total weight of the composition.

19. The composition according to claim 9, wherein solvent C of the solvent mixture is present in an amount of from 5% to 40% by weight relative to the total weight of the composition.

20. The flavored product according to claim 13, wherein the composition is present in an amount of from 0.01% to 1% by weight relative to the total weight of the flavored product.
